(19) United States
(12) Patent Application Publication
(10) Pub. No.: US 2017/0340861 A1
(43) Pub. Date: Nov. 30, 2017

(54) CONDUCTOR CABLES FOR USE IN STEERABLE DEVICES

(71) Applicant: General Electric Company, Schenectady, NY (US)

(72) Inventor: Douglas Glenn Wildes, Ballston Lake, NY (US)

(21) Appl. No.: 15/163,312

(22) Filed: May 24, 2016

(51) Int. Cl. A61M 25/01; A61B 1/00

(52) U.S. Cl. CPC A61M 25/0113; A61M 25/0138; A61B 1/00078; A61M 2207/00

(57) ABSTRACT

A multi-conductor cable for use in a catheter is described. The conductor-cable is isotropically flexible to allow catheter steering, but is also longitudinally stiff enough to push and pull the entire cable through the catheter lumen. In one implementation, the multi-conductor cable is bundled for most of its length, to provide longitudinal stiffness and support pushing and pulling the cable through a catheter lumen. However, in one such implementation, the portion of the cable within a steerable section of the catheter is unbundled and divided into individual conductors or groups that are flexible enough that they do not bias or otherwise interfere with the steering of the catheter. In one such approach, a spring wire or other stiffening element is added to the cable at that location to preserve or enhance longitudinal stiffness (tension and compression) of the cable through the unbundled flexible section.

### BACKGROUND

The subject matter disclosed herein relates to the use of cables and conductive components within tools or instruments designed to be navigated through a patient or object during a procedure or inspection (e.g., steerable tools or instruments, such as catheters, endoscopes, laparoscopes, etc.).

As medical imaging technologies have matured, it has become possible to combine the use of medical imaging techniques with interventional procedures or techniques. For example, such technologies may be employed when performing minimally invasive procedures, guiding or employing catheters for cardiac or surgical procedures, or otherwise steering a tool to a desired location within the patient. In some instances, imaging functionality, such as ultrasound imaging functionality, may be provided on the tool itself and this imaging functionality may be used to facilitate another procedure or may be the focus of the procedure itself, e.g., to obtain localized images from within the patient.

In many instances, steerable tools, such as steerable catheters, may be employed to position the tool or instrument, including ultrasound imagers, within the patient. In such instances, the catheters are typically steered by pulling on, and thereby compressing, one side of the catheter tube using pull wires internal to the tube or a secondary tube internal to the primary tube. As a catheter is repeatedly steered and straightened, the length of the catheter repeatedly changes. Cable running through the lumen of the catheter (such as cables or conductors associated with the operation of the tool or imager at the tip of the catheter) must accommodate that change, typically by sliding back and forth through the lumen.

However, as the number of electrical conductors, optical fibers, capillary tubes, and other conductive elements through the lumen increases and/or the diameter of the catheter decreases, it may become more difficult to move or position these elements within the catheter lumen. For example, in some instances the individual conductors are not stiff enough to push the associated cable through the catheter shaft when the tip section is steered and compressed.

One conventional solution is to tightly bundle the conductors so that the bundle is stiff enough to push through the lumen. However, the stiff bundle in turn reduces the steerability (i.e., flexibility) of the catheter. That is, there is an inherent conflict between a cable that is loose enough for easy bending but stiff enough to push itself through lumen. Further, if the bundle is not cylindrical and isotropic (e.g., if the cable is a stack of flat flex circuits), then it bends more easily in some directions than in others, which biases the steering of the catheter. Further, if the fine conductors are not bundled, then as the catheter is steered and compressed, the conductors tend to bunch together and kink. Over repeated cycles, the kinks become cracks and the conductors fail.

The preceding examples are provided in a medical context so as to give a useful frame-of-reference and useful example. However, it should be appreciated that such issues may be present in non-medical contexts as well, such as in equipment inspection (optical or ultrasound-based) as well as other industrial or commercial contexts.

### BRIEF DESCRIPTION

In one implementation, a steerable device is provided. In accordance with this implementation, the steerable device includes one or more deflection regions and a lumen running through the steerable device. The steerable device also includes a multi-conductor cable configured to move within the lumen. Portions of the multi-conductor cable configured to be positioned in the deflection regions of the steerable device include one or more stiffening elements and two or more conductor elements unbound from one another and moving at least partially independent of one another.

In another implementation a steerable device is provided. In accordance with this implementation, the steerable device includes one or more deflection regions. The steerable device also includes a flexible circuit within a lumen of the steerable device. The flexible circuit, within the deflection regions, includes at least one slit running substantially parallel to conductive elements within the flexible circuit. The steerable device also includes at least one stiffening element disposed within each deflection region.

In a further implementation, a method for improving steerability of a steerable device is provided. In accordance with this implementation, for one or more deflection regions of a steerable device, two or more conductive elements of a multi-conductor structure running through the respective deflection region are physically separated. One or more stiffening elements are provided along with the multi-conductor structure in the one or more deflection regions.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic view of an implementation of components of a diagnostic or therapy system, in accordance with aspects of the present disclosure;

FIG. 2 depicts an example of a steerable device (e.g., a catheter) suitable for use with a present multi-conductor cable, in accordance with aspects of the present disclosure;

FIG. 3 depicts a deflection region of a catheter shaft having unbundled conductive elements, in accordance with aspects of the present disclosure;

FIG. 4 depicts a slitted flex circuit, in accordance with aspects of the present disclosure;

FIG. 5 depicts a picture of a prototype slitted flex circuit, in accordance with aspects of the present disclosure; and

FIG. 6 depicts another view of the slitted flex circuit of FIG. 5 outside the catheter tubing.

### DETAILED DESCRIPTION

One or more specific implementations will be described below. In an effort to provide a concise description of these implementations, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements.

A multi-conductor cable for use in a steerable or interventional device (e.g., a catheter, endoscope, laparoscope, and so forth) is described herein. The multi-conductor cable is isotropically flexible to allow steering of the device, but is also longitudinally stiff enough to push and pull the entire cable through a lumen of the device. In one implementation, the multi-conductor cable is bundled for most of its length, to provide longitudinal stiffness and support pushing and pulling the cable through the lumen. However, in one such implementation, the portion of the cable within a steerable section of the steerable device is unbundled and divided into individual conductors or groups that are flexible enough that they do not bias or otherwise interfere with the steering of the device. In one such approach, a spring wire or other stiffening element is added to the cable at that location to preserve or enhance longitudinal stiffness (tension and compression) of the cable through the unbundled flexible section. In such an approach, the spring wire is anchored in the bundled cable both proximal and distal to the unbundled section. When employed in a catheter (or other steerable device), the result is a catheter that that has omnidirectional steering (e.g., is steerable in multiple directions) and is not biased in certain directions due to the effects of the internal conductive elements.

Though certain of the following examples are provided in a medical context so as to provide a useful frame-of reference, it should be appreciated that these examples are not intended to be limiting and are provided merely to facilitate explanation of the present approaches. Indeed, the present approaches may be useful in other contexts including, but not limited industrial inspection and quality control, security inspection and observation, and other industrial, commercial, or security applications where a device may be employed that is steered within or through an object or subject, such as for optical or ultrasound inspection.

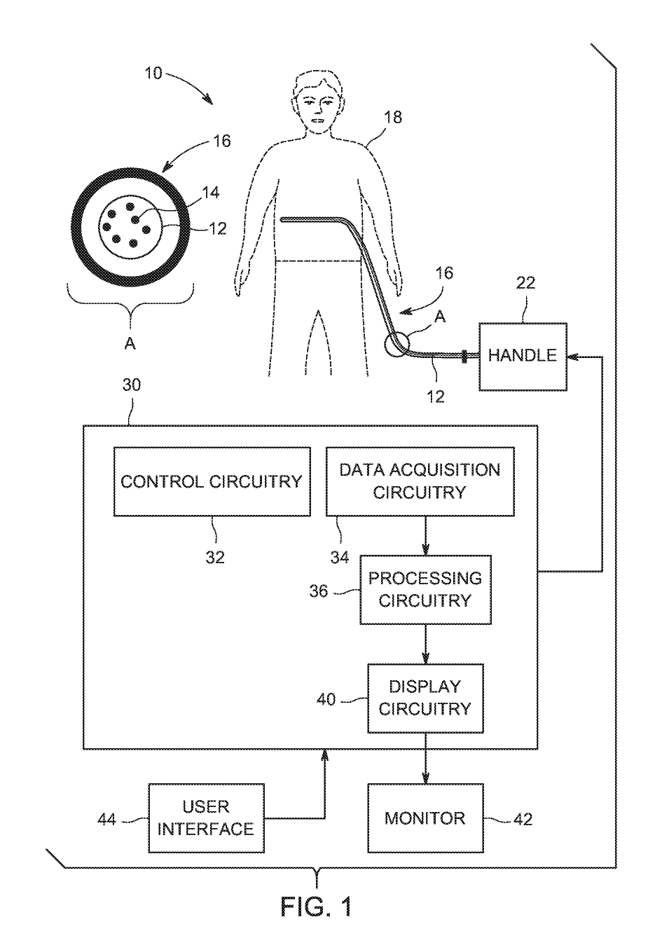

With the preceding in mind, FIG. 1 depicts an interventional (e.g., minimally invasive diagnostic or therapy) system 10 employing a steerable device in the form of a catheter 16 in which the cable described herein may be employed. In the depicted example, a cable 12 is shown schematically in part as being a multi-conductor cable 12 having multiple, separate and distinct conductor elements 14 that run within the catheter 16 and an operator interface 22 (e.g., a probe, handle, or other user-manipulated aspects) used to operate the catheter 16 and/or the cable 12 within the catheter 16. As used herein, the conductive elements 14 may include, but are not limited to: wires, coax cables (coax), optical fibers, flex circuits, and so forth. In some instances the cable 12 may include lumens or tubing (e.g., capillary tubes) suitable for the passage of fluids or gases that may be utilized in a given procedure.

In one implementation, the interventional system 10 provides imaging functionality using the conductor elements 14, such as a phased-array ultrasound imager (e.g., transducer elements and circuitry) that can be positioned and moved at the tip of the catheter 16 that is inserted and navigated within a patient 18. It should be appreciated though that other functionality, including surgical functionality (e.g., remote operation of surgical instrumentation) and/or treatment functionality (e.g., application of heat, cold, ablative radiation or energy, and so forth) may be provided using the conductive elements 14 in addition to or instead of such imaging functionality.

In an imaging implementation, as shown in FIG. 1, other components that may be present in the overall system 10, typically include some type of imaging transducer (e.g., a phased-array ultrasound transducer array or other elements) within or affixed to the catheter 16 which generates signals that can be converted to image data. The transducer, when present, may be operated by system control circuitry 30 which controls various aspects of the imager operation and acquisition and processing of the image data. In the depicted generalized example, the system control circuitry 30 includes control circuitry 32 useful in operating the imaging transducer when present in the catheter. For example, the control circuitry 32 may include circuitry for transmitting ultrasound, receiving and amplifying ultrasound, and/or otherwise operating the imaging transducer within the catheter 16. Acquisition of the image data or signals may be facilitated by data acquisition circuitry 34. For digital systems, the data acquisition circuitry 34 may perform a wide range of initial processing functions. The data may then be transferred to data processing circuitry 36 where additional processing, analysis, and or image generation are performed. For the various digital imaging systems available, the data processing circuitry 36 may perform substantial reconstruction and/or analyses of data, ordering of data, sharpening, smoothing, feature recognition, and so forth.

The processed image data may be forwarded to display circuitry 40 for display at a monitor 42 for viewing and analysis. While operations may be performed on the image data prior to viewing, the monitor 42 is at some point useful for viewing reconstructed images derived from the image data collected. The images may also be stored in short or long-term storage devices which may be local to the system 10, such as within the system control circuitry 30, or remote from the system 10, such as in picture archiving communication systems. The image data can also be transferred to remote locations, such as via a network.

For simplicity, certain of the circuitry discussed above, such as the control circuitry 32, the data acquisition circuitry 34, the processing circuitry 36, and the display circuitry 40, are depicted and discussed as being part of the system control circuitry 30. Such a depiction and discussion is for the purpose of illustration only, however, and is intended to merely exemplify one possible arrangement of this circuitry in a manner that is readily understandable. Those skilled in the art will readily appreciate that in other implementations the depicted circuitry may be provided in different arrangements and/or locations. For example, certain circuits may be provided in different processor-based systems or workstations or as integral to different structures, such as imaging workstations, system control panels, and so forth, which functionally communicate to accomplish the techniques described herein.

The operation of the interventional system 10 may be controlled by an operator via a user interface 44 which may include various user input devices, such as a mouse, keyboard, touch screen, and so forth. Such a user interface 44 may be configured to provide inputs and commands to the system control circuitry 30, as depicted. Moreover, it should also be noted that more than a single user interface 44 may be provided.

While the preceding description has generally been in the context of providing some form of imaging functionality with respect to the diagnostic or therapy system 10, as noted above the system 10 may instead of, or in addition to, provide treatment (e.g., ablation) and/or surgical functionality. In such implementations, the system 10 may include additional or alternative circuitry to provide the needed functionality and/or the described circuitry may support the additional functions. Similarly, in non-medical contexts (e.g., industrial or security contexts) the system 10 may provide appropriate imaging or other functionality in the context of a suitable application.

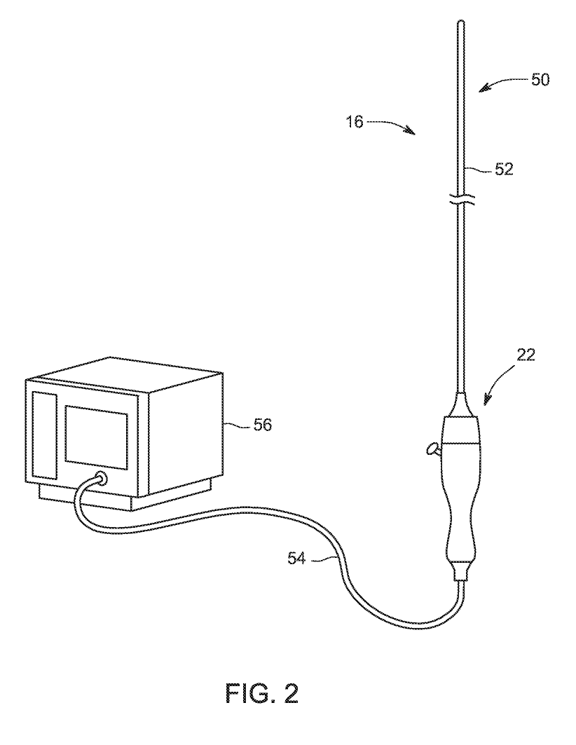

While FIG. 1 is primarily a schematic or block-type view of a diagnostic or therapy system 10 suitable for use of the cable 12 discussed herein, FIG. 2 depicts one example of a catheter-based system as it may appear in a real-world setting. In this example, the catheter 16 is suitable for insertion into and navigation through the vasculature of a patient. The depicted catheter 16 includes a distal end or tip 50 in which one or more instruments or devices may be positioned or moved and which, during operation, utilize conductors 14 (FIG. 1) to receive control signals and/or transmit data. The instrumentation and conductors 14 may be moved within a shaft 52 which connects the tip 50 with a handle assembly 22 that may be used to manipulate and operate the catheter 16. As discussed herein, the shaft 52 may be flexible to facilitate being steered within the patient and the handle 22, via one or more control structures, may operate one or more pull wires in the shaft 52 to bend the shaft in various directions in response to the commands of a user. In certain instances, the handle 22 may communicate, such as via a cable 54, with an operator console 56 that allows a user to control certain aspects of the catheter function and operation, as described more generally with respect to FIG. 1.

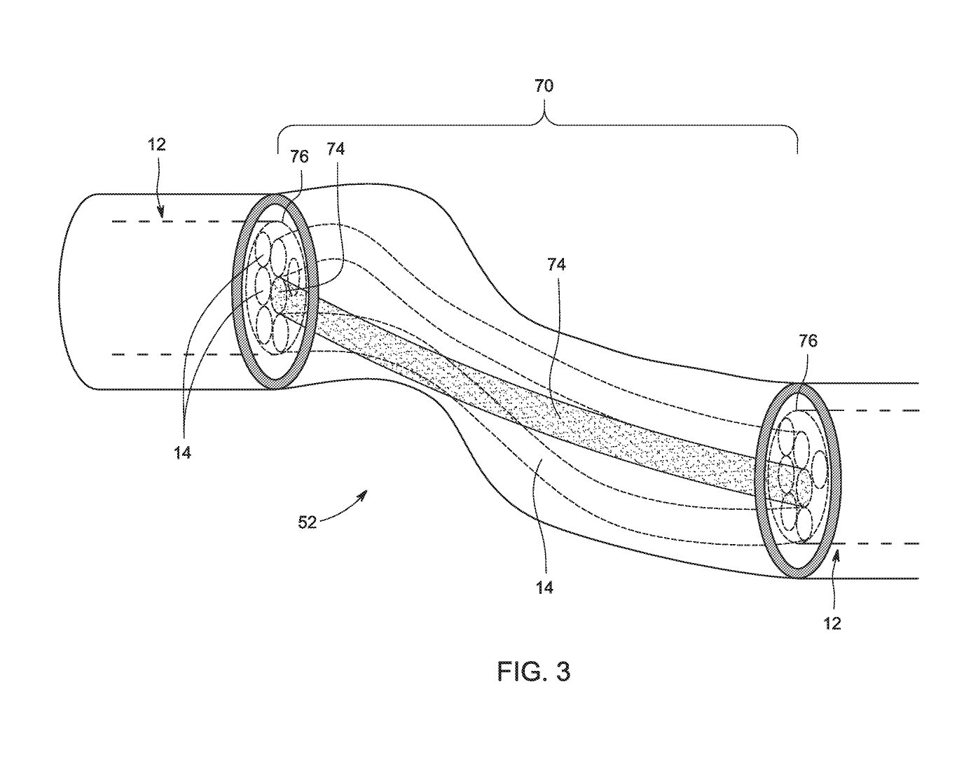

As discussed herein, to facilitate flexing of the shaft 52 and movement of the conductive elements 14, various approaches are contemplated. For example, turning to FIG. 3, in one implementation, the conductive elements 14, bundled as a multi-conductor cable 12, run through the shaft 52 of the catheter. In those regions of the shaft 52 where flexion is not an issue (e.g., straight or non-flexing portions of the shaft 52), the conductive elements 14 may be bunched or bound together (i.e., move as a unit) in the cable 12, such as in a fitted inner tubing 76 (e.g., heat shrink tubing). Bundling of the conductive elements 14 in this manner provides longitudinal stiffness, which allows the cable 12 to be easily pushed and pulled within the catheter lumen.

However, as depicted in FIG. 3, the shaft 52 may also include flexible deflection regions 70 through which the conductive elements 14 pass but through which bundling of these conductive elements 14 may be undesirable or infeasible due to the need to flex the shaft 52 at the deflection region 70 to allow the catheter 16 to be steered. As shown in the depicted example, the sheath (fitted inner tubing) 76 or other bundling mechanism may be released or removed in this deflection region 70 (i.e., the conductive elements 14 are unbundled) so as to allow the conductive elements 14 to move freely and independently relative to one another in this region, thus reducing the forces that might otherwise inhibit flexing of the shaft 52 at deflection region 70. In particular, when unbundled, the conductive elements 14 are flexible enough that they do not bias or otherwise interfere with the steering of the catheter 16. As a result of this unbundling of the conductive wires in the deflection region 70, the catheter 16 is steerable in multiple directions (i.e., omnidirectional steering) without bias from the conductive wires interfering.

However, as will be appreciated, longitudinal stiffness may be reduced or lost within the deflection regions 70 where the conductive elements 14 are unbundled. Therefore, in certain implementations a spring wire(s) 74 or other longitudinal stiffening element(s) is added to the cable 12 in these regions to preserve longitudinal stiffness (tension and compression) of the cable 12 through the unbundled deflection region 70. The spring wire 74 is stiff enough to push the cable 12 through the catheter lumen, but flexible enough to allow easy steering. In such an implementation, the spring wire 74 may be anchored in the bundled cable 12 both proximal and distal to the unbundled deflection region 70. As will be appreciated, though longitudinally stiff, the spring wire (or other stiffening element) is laterally flexible so as to accommodate deflection within the region 70. In general, the stiffening element (in the various embodiments discussed herein) is stiffer than the unbundled regions of the multi-conductor cable in the longitudinal direction, but is more flexible (in the lateral direction) than the bundled (i.e., stiff) regions of the multi-conductor cable. The stiffening element may or may not be more flexible in the lateral direction than the unbundled portions of the cable. In one implementation, the stiffening element 74 is a 0.2 mm-0.4 mm (e.g., 0.25 mm diameter) diameter spring-tempered stainless steel element (e.g., a wire) that transfers longitudinal forces (e.g., tension and compression) across unbundled deflection region 70 without buckling and without allowing the conductive elements 14 to kink or become damaged. Depending on the implementations, stiffening elements of other diameters, sizes, and/or stiffness, may be employed.

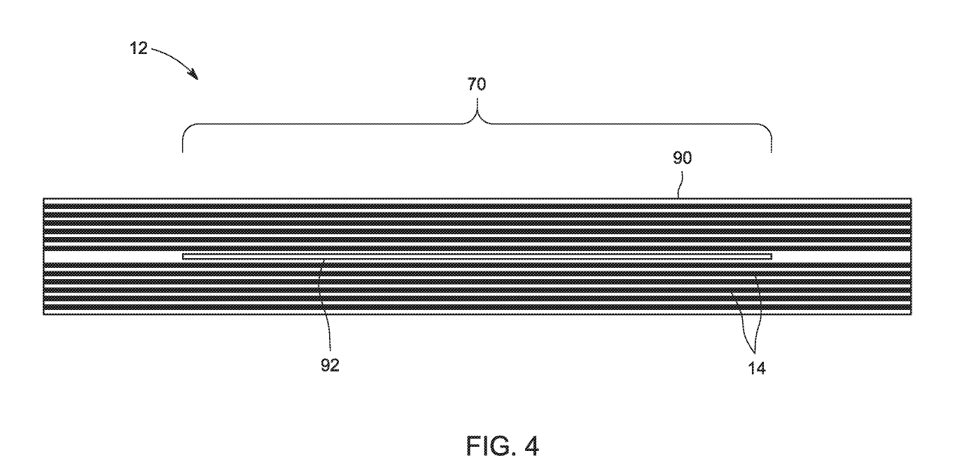

While FIG. 3 describes one implementation, in other implementations the conductive elements 14 may be provided as part of an existing flexible cable or flexible circuit (e.g., a flex circuit). For example, turning to FIG. 4 if the conductors 14 are flex circuits 90 or flat ribbonized wires, coax, or optical fibers, they will tend to be flexible for out-of-plane bending but very stiff for in-plane bending. If omnidirectional bending (i.e., omnidirectional catheter steering) is needed, the flex circuits 90 or ribbonized conductor groups are slit (slit 92) into two or more strips of reduced in-plane width and lateral stiffness for the unbundled length (i.e., in deflection region 70) of the cable 12. The reduced-width strips will be more flexible in-plane and will be more able to move and reorient within the catheter lumen to allow steering to large angles with minimum resistance.

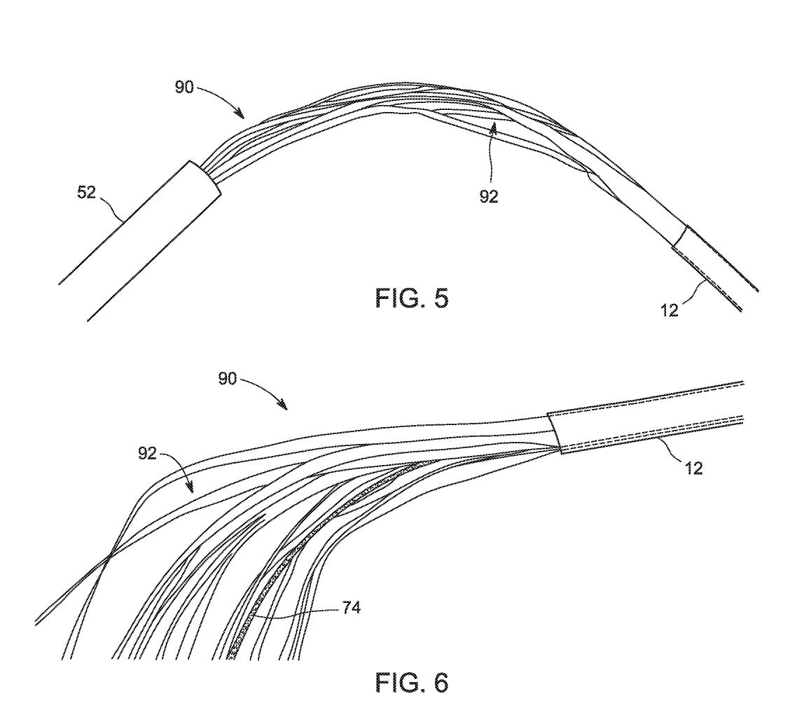

With this in mind, FIGS. 5 and 6 depict pictures of a prototype implementation of one such flex circuit approach. With respect to these prototypes, a scalpel was used to slit each of a set of ˜1.2 m long flex circuits 90 for a distance of ˜8.5 cm near one end. A ˜13 cm length of a 0.25 mm diameter spring-tempered stainless steel wire 74 was flattened for a distance of ˜1 cm at each end, and the ends were attached (with epoxy) to ˜2 mm wide strips of flex circuit material 90. In other implementations for greater reliability the ends could instead be welded to strips of thin metal. The non-slitted portions of the long flex circuits 90 were aligned and bundled together with heat-shrink tubing to form the cable 12. The spring wire 74 was included in the middle of the stack of flex circuits 90, aligned with the slits 92, with the flex material attached to the ends of the spring wire 74 included in and thereby anchored to the bundled stack of flex circuits at both ends of the slits 92.

The resulting cable 12 was pulled through a length of 2.5 mm ID plastic tubing (equivalent to a catheter shaft 52 and lumen). Manual (non-quantitative) experiments verified that the entire cable 12 could be pushed and pulled through the catheter shaft 52 by pushing and pulling on the end nearest the slits 92, with no bunching or kinking of the flex circuits 90, even with moderate friction between the cable 12 and the catheter shaft 52. Omnidirectional flexibility of the slitted cable, and of the catheter shaft 52 in the region containing the slitted cable, was verified. In this manner loose (i.e., not bundled) strips of slitted flex circuit 90 in the deflection region 70 of a catheter shaft 52 may be employed to allow omnidirectional flexibility of the deflection region 70. To facilitate movement of the slitted flex-circuit where unbundled, a spring wire or other stiffening element 74 provides tensile and compressive strength.

Technical effects of the invention include a catheter having unbundled conductive elements, including in the form of a slitted flex circuit, within a deflection region. Within the deflection region, a stiffening element 74 may be included to provide longitudinal stiffness.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A steerable device, comprising:
one or more deflection regions of the steerable device;
a lumen running through the steerable device;
a multi-conductor cable configured to move within the lumen, wherein portions of the multi-conductor cable configured to be positioned in the deflection regions of the steerable device comprise:
one or more stiffening elements; and
two or more conductor elements unbound from one another and moving at least partially independent of one another.

2. The steerable device of claim 1, wherein, outside the deflection regions, the two or more conductor elements of the multi-conductor cable are bound together.

3. The steerable device of claim 1, wherein the multi-conductor cable is isotropically flexible within the deflection regions.

4. The steerable device of claim 1, wherein the steerable device comprises one of a catheter, an endoscope, or a laparoscope.

5. The steerable device of claim 1, wherein each stiffening element has a diameter between about 0.2 mm to about 0.4 mm.

6. The steerable device of claim 1, wherein the multi-conductor cable comprises one or more of wire or wires, coax cable, optical or other optic fibers, capillary tubes, or flex circuitry.

7. The steerable device of claim 1, wherein each stiffening element is anchored on either end of the respective deflection region in which the stiffening element is located.

8. The steerable device of claim 1, wherein each stiffening element is stiffer in the longitudinal direction than regions of the multi-conductor cable that are unbundled and more flexible in the lateral direction than regions of the multi-conductor cable that are bundled.

9. A steerable device, comprising:
one or more deflection regions;
a flexible circuit within a lumen of the steerable device, wherein the flexible circuit, within the deflection regions, comprises at least one slit running substantially parallel to conductive elements within the flexible circuit; and
at least one stiffening element disposed within each deflection region.

10. The steerable device of claim 9, wherein respective slits in the flexible circuit do not extend beyond the respective deflection regions.

11. The steerable device of claim 9, wherein the flexible circuit is flexible in-plane within the deflection regions relative to the in-plane flexibility exhibited outside the deflection regions.

12. The steerable device of claim 9, wherein each stiffening element comprises a spring wire.

13. The steerable device of claim 9, wherein each stiffening element has a diameter between about 0.2 mm to about 0.4 mm.

14. The steerable device of claim 9, wherein each stiffening element comprises stainless steel.

15. The steerable device of claim 9, wherein each stiffening element is anchored on either end of the respective deflection region in which the stiffening element is located.

16. The steerable device of claim 9, wherein each stiffening element is stiffer in the longitudinal direction of a respective deflection region than regions of the flexible circuit that are slit.

17. A method for improving steerability of a steerable device, comprising:
for one or more deflection regions of a steerable device, physically separating two or more conductive elements of a multi-conductor structure running through the respective deflection region; and
providing one or more stiffening elements along with the multi-conductor structure in the one or more deflection regions.

18. The method of claim 17, wherein the multi-conductor structure comprises a multi-conductor cable and physically separating the two or more conductive elements comprises unbundling the two or more conductive elements from the multi-conductor cable.

19. The method of claim 17, wherein the multi-conductor structure comprises a flexible circuit and physically separating the two or more conductive elements comprises slitting the flexible circuit to form one or more slits at the location of the respective deflection regions.

20. The method of claim 17, wherein providing one or more stiffening elements comprises providing one or more spring wires in the one or more deflection regions.
